(19) United States
(12) Patent Application Publication
(10) Pub. No.: US 2010/0203516 A1
(43) Pub. Date: Aug. 12, 2010

(54) DETECTING ANALYTES USING BOTH AN OPTICAL AND AN ELECTRICAL MEASUREMENT METHOD

(75) Inventors: Colin Campbell, Edinburgh (GB); Peter Ghazal, Edinburgh (GB); John Beattie, Edinburgh (GB); Andrew Mount, Edinburgh (GB); Till Bachmann, Edinburgh (GB)

Correspondence Address: Joseph R. Baker, APC;Gavrilovich, Dodd & Lindsey LLP, 4660 La Jolla Village Drive, Suite 750, San Diego, CA, 92122, US

(73) Assignee: ITI SCOTLAND LIMITED, Glasgow (GB)

(21) Appl. No.: 12/524,562

(22) PCT Filed: Jan. 25, 2008
(86) PCT No.: PCT/EP2008/050910

§ 371 Date: Mar. 5, 2010

(30) Foreign Application Priority Data
Jan. 25, 2007 (GB) 0701444.2

(51) Int. Cl. C12Q 1/68; G01N 21/00; G01N 27/26; G01N 33/53; G01N 33/68

(52) U.S. Cl. 435/6; 436/164; 205/792; 436/94; 435/7.1; 436/86

(57) ABSTRACT

Provided is a method for detecting an analyte, wherein the analyte is labelled with one or more labels relatable to the analyte, which method comprises: a) performing an optical detection method on the labelled analyte to obtain optical data from the one or more labels; b) performing an electrical detection method on the labelled analyte to obtain electrical data from the one or more labels; and c) determining the identity and/or quantity of the analyte from both the optical and electrical data. Further provided is a method for detecting a plurality of analytes, wherein the each different analyte is labelled with one or more different labels relatable to the analyte, which method comprises: a) performing an optical detection method on a plurality of labelled analytes to obtain optical data from the labels; b) performing an electrochemical detection method on the plurality of labelled analytes to obtain electrical data from the labels; and c) determining the identity and/or quantity of the plurality of analytes from both the optical and electrical data.

This invention relates to methods for detecting an analyte or a plurality of analytes, particularly for detecting proteins or DNA. Specifically this invention relates to methods for detecting an analyte comprising both optical and electrical detection of labelled analytes.

Methods for detecting analytes are well known in the field of biochemical analysis. In traditional methods the analyte is labelled, usually with a fluorescent label, which can be detected, for example by fluorescence detection, in order to identify the analyte.

In the past few years in the field of DNA detection, nanoparticles have been used as the labels. These labels will potentially work for any system that permits labelling and involves binding, thus may be useful in a live cell system, as well as proteins and nucleic acids. The nanoparticles have been found to overcome a number of limitations of fluorescent labels including cost, ease of use, sensitivity and selectivity (Fritzsche W, Taton T A, Nanotechnology 14 (2003) R63-R73 “Metal nanoparticles as labels for heterogeneous, chip-based DNA detection”). Nanoparticles have been used in a number of different DNA detection methods including optical detection, electrical detection, electrochemical detection and gravimetric detection (Fritzsche W, Taton T A, Nanotechnology 14 (2003) R63-R73 “Metal nanoparticles as labels for heterogeneous, chip-based DNA detection”). The use of gold nanoparticles in the detection of DNA hybridization based on electrochemical stripping detection of the colloidal gold tag has been successful (Wang J, Xu D, Kawde A, Poslky R, Analytical Chemistry (2001), 73, 5576-5581 “Metal Nanoparticle-Based Electrochemical Stripping Potentiometric Detection of DNA hybridization”). The use of semiconductor nanocrystals, also called quantum dots, and gold nanoparticles have also been successfully used as fluorescent labels for DNA hybridization studies (West J, Halas N, Annual Review of Biomedical Engineering, 2003, 5: 285-292 “Engineered Nanomaterials for Biophotonics Applications: Improving Sensing, Imaging and Therapeutics”).

Despite the advantages discovered by using nanoparticles in DNA detection methods instead of the previous fluorescent labels, there is still a need to improve the sensitivity and selectivity of the detection methods. Whilst each detection method has a certain degree of sensitivity and selectivity, they each have different limitations and produce different inaccuracies.

In addition to the need for improved sensitivity and selectivity in analyte detection methods there is also a growing need for quick, cheap and simple detection methods, particularly for DNA.

It is an object of this invention to overcome the problems associated with the above prior art. In particular, it is an aim of this invention to provide a method for detecting an analyte with improved sensitivity and selectivity which is also quick, cheap and simple to carry out.

Accordingly, the present invention provides a method for detecting an analyte, wherein the analyte is labelled with one or more labels relatable to the analyte, which method comprises:
a) performing an optical detection method on the labelled analyte to obtain optical data from the one or more labels;
b) performing an electrical detection method on the labelled analyte to obtain electrical data from the one or more labels; and
c) determining the identity and/or quantity of the analyte from both the optical and electrical data.

The present invention also provides a method for detecting a plurality of analytes, wherein the each different analyte is labelled with one or more different labels relatable to the analyte, which method comprises:
a) performing an optical detection method on a plurality of labelled analytes to obtain optical data from the labels;
b) performing an electrochemical detection method on the plurality of labelled analytes to obtain electrical data from the labels; and
c) determining the identity and/or quantity of the plurality of analytes from both the optical and electrical data.

The present invention is distinguished by the fact that both optical and electrical detection methods are carried out on the labelled analyte or plurality of labelled analytes. The present inventors have surprisingly discovered that both optical and electrical detection methods can be carried out on a labelled analyte or plurality of labelled analytes if the optical method is carried out first followed by the electrical method.

The inventors have also surprisingly discovered that in a preferred embodiment the use of labels which are suitable for optical and electrical detection allows, after optical detection, the labelled analytes to be in a state that can be successfully used in electrical detection.

The advantages of the methods of the present invention are that they improve sensitivity and selectivity of the results. When a plurality of different analytes is to be detected, the present method increases the accuracy and number of the analytes detected. These advantages result directly from the use of both the optical data from the optical detection method and the electrical data from the electrical detection method to determine the identity and/or quantity of the analyte or plurality of analytes.

Whilst it is known in the art to use optical and electrical detection methods for analytes separately, it has never been taught or even suggested to use both methods on a labelled analyte or plurality of labelled analytes.

In a preferred aspect of the invention, the one or more labels are suitable for optical and electrical detection and the one or more labels used in step (a) are the same as the one or more labels used in step (b) of the method. This more readily allows the data from both the optical and electrical methods to be used to determine the identity and/or quantity of analyte or plurality of analytes in one sample.

In an alternative aspect of the invention, the one or more labels in step (a) are suitable for optical detection and the one or more labels in step (b) are suitable for electrical detection and the one or more labels in step (a) are different from the one or more labels in step (b). This is advantageous because it provides more data when the optical detection and electrical detection are carried out on separate labels.

The sensitivity and selectivity of the method of the present invention is improved significantly compared to carrying out either an optical detection method or an electrical detection method.

The methods of the present invention are also quick, cheap and simple to carry out.

The present invention will be described in further detail with reference to the accompanying Figures, in which:

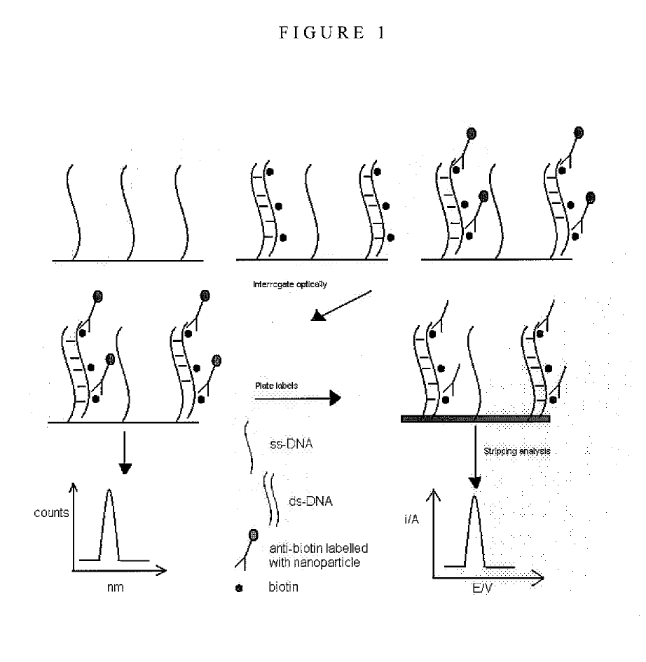

FIG. 1 shows a schematic representation of the method of the present invention. The method may be employed for detecting any analyte, including DNA or RNA.

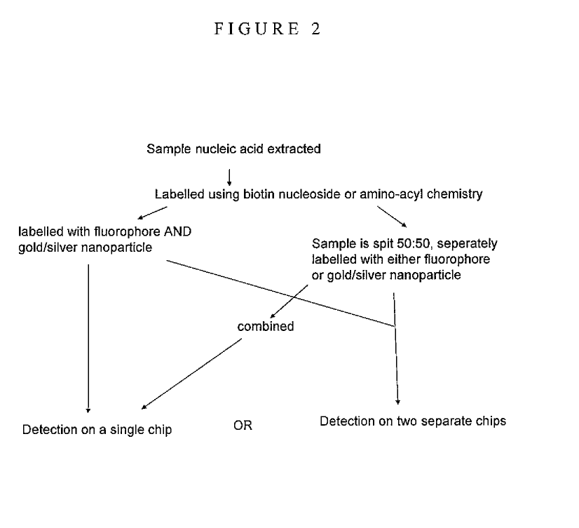

FIG. 2 shows a flow diagram of different routes for labelling the analyte(s) with a different label for step (a) and step (b).

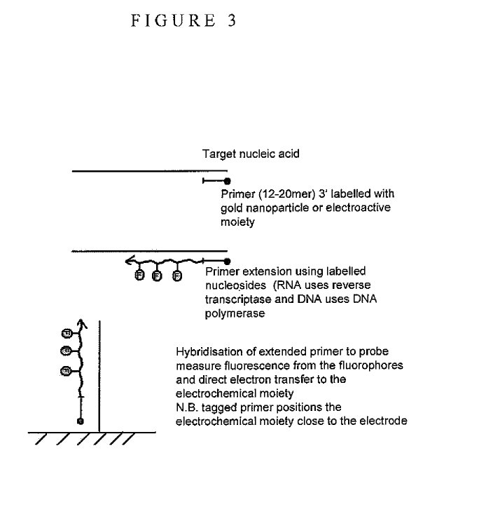

FIG. 3 shows a schematic representation of a method for labelled the analyte(s) when they are nucleic acids with a different label for step (a) and step (b).

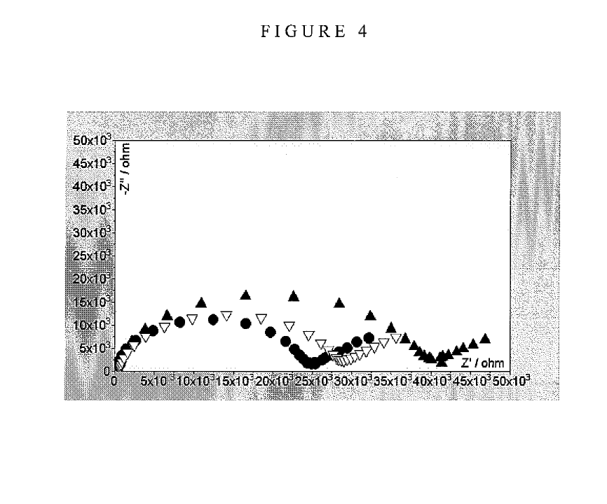

FIG. 4 shows a Nyquist plot of electrode with probe only (black circles), probe hybridised with 100 nM complementary target (black triangles) and probe after removal of target (white triangles).

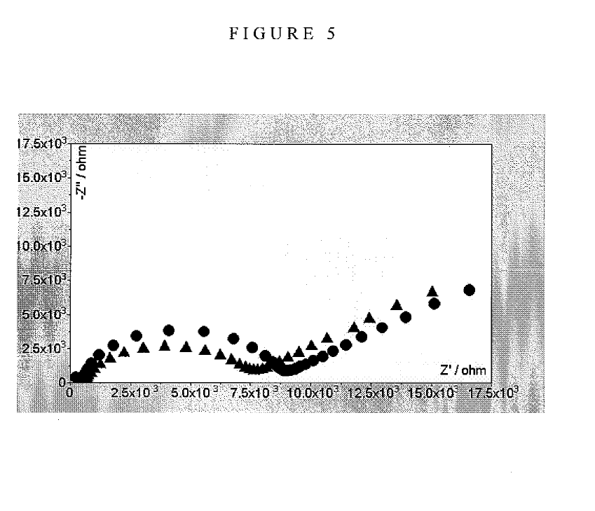

FIG. 5 shows a Nyquist plot of electrode with probe only (black circles) and after hybridisation with 100 nM non-complementary target (black triangles).

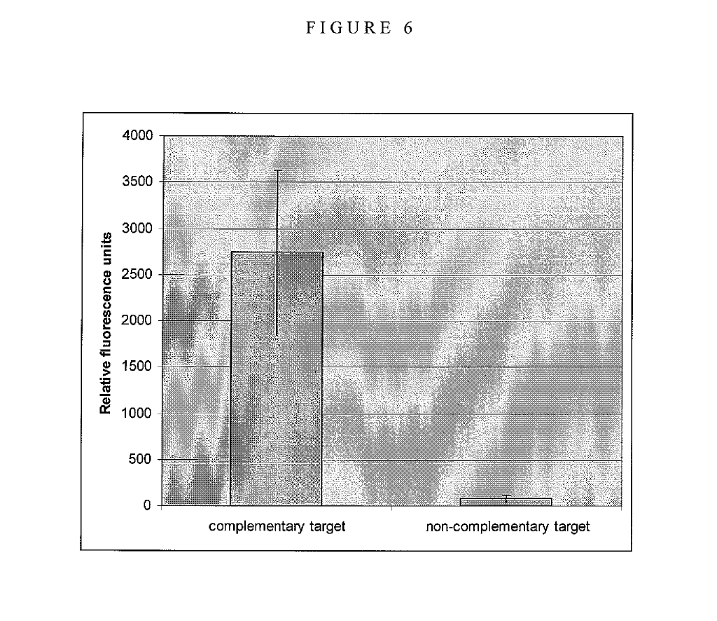

FIG. 6 shows fluorescence measured from electrodes hybridised with complementary target or non-complementary target. Error bars show the standard deviation of pixel intensity across the electrode.

The analyte for detection in the present method preferably comprises one or more compounds selected from a cell, a protein, a polypeptide, a peptide, a peptide fragment, an amino acid, DNA and RNA. The method of the present invention is particularly useful for DNA and RNA detection.

The method of the present invention may be used to detect one analyte or a plurality of different analytes. When the method is used to detect a plurality of different analytes, each different analyte may be labelled with one or more different labels relatable to the analyte. Alternatively, multiple analytes may be detected by spatial separation, such as by arraying a set of probes for the analytes on a surface. Detection of a plurality of different analytes is also known as multiplexing.

### Label

In a preferred embodiment of the present invention, the one or more labels are selected from nanoparticles, single molecules, intrinsic components of the target such as specific nucleotides or amino acids, and chemiluminescent enzymes. Suitable chemiluminescent enzymes include HRP and alkaline phosphatise.

In the embodiment of the present invention wherein the label or labels used in step (a) are different from the label or labels used in step (b) of the method, the label(s) used in step (a) may be for example fluorophores and the labels used in step (b) may be nanoparticles, single molecules and chemiluminescent enzymes.

Preferably, the labels are nanoparticles. Nanoparticles are particularly advantageous in the embodiment of the present invention where the label(s) used in step (a) are the same as the label(s) used in step (b) because they operate successfully in both optical and electrical detection methods. The proximity of the nanoparticles to the surface is not especially important, which makes the assay more flexible. In a preferred embodiment the nanoparticles comprise a collection of molecules because this gives rise to greater signal in optical and electrical detection methods than when single molecules are used.

Preferably the nanoparticles are selected from metals, metal nanoshells, metal binary compounds and quantum dots. Examples of preferred metals or other elements are gold, silver, copper, cadmium, selenium, palladium and platinum. Examples of preferred metal binary and other compounds include CdSe, ZnS, CdTe, CdS, PbS, PbSe, HgI, ZnTe, GaAs, HgS, CdAs, CdP, ZnP, AgS, InP, GaP, GaInP, and InGaN.

Metal nanoshells are sphere nanoparticles comprising a core nanoparticle surrounded by a thin metal shell. Examples of metal nanoshells are a core of gold sulphide or silica surrounded by a thin gold shell.

Quantum dots are semiconductor nanocrystals, which are highly light-absorbing, luminescent nanoparticles (West J, Halas N, Annual Review of Biomedical Engineering, 2003, 5: 285-292 “Engineered Nanomaterials for Biophotonics Applications: Improving Sensing, Imaging and Therapeutics”). Examples of quantum dots are CdSe, ZnS, CdTe, CdS, PbS, PbSe, HgI, ZnTe, GaAs, HgS, CdAs, CdP, ZnP, AgS, InP, GaP, GaInP, and InGaN nanocrystals.

Any of the above labels may be attached to an antibody, see for example FIG. 1 which shows an anti-biotin labelled with a nanoparticle.

The size of the labels is preferably less than 200 nm in diameter, more preferably less than 100 nm in diameter, still more preferably 2-50 nm in diameter, still more preferably 5-50 nm in diameter, still more preferably 10-30 nm in diameter, most preferably 15-25 nm.

When the method of the present invention is for detecting a plurality of analytes, each different analyte is labelled with one or more different labels relatable to the analyte. In this aspect of the invention, the labels may be different due to their composition and/or type. For example, when the labels are nanoparticles the labels may be different metal nanoparticles. When the nanoparticles are metal nanoshells, the dimensions of the core and shell layers may be varied to produce different labels. Alternatively or in addition, the labels have different physical properties, for example size, shape and surface roughness. In one embodiment, the labels may have the same composition and/or type and different physical properties.

The different labels for the different analytes are preferably distinguishable from one another in the optical detection method and the electrical detection method. For example, the labels may have different frequencies of emission, different scattering signals and different oxidation potentials.

### Labelling the Analyte

In a preferred embodiment of the present invention the method comprises a further step before step (a) of labelling the analyte with one or more labels to form the labelled analyte.

The means for labelling the analyte are not particularly limited and many suitable methods are well known in the art. For example, when the analyte is DNA or RNA it may be labelled by enzymatic extension of label-bound primers, post-hybridization labelling at ligand or reactive sites or “sandwich” hybridization of unlabelled target and label-oligonucleotide conjugate probe (Fritzsche W, Taton T A, Nanotechnology 14 (2003) R63-R73 “Metal nanoparticles as labels for heterogeneous, chip-based DNA detection”).

Many different methods are known in the art for conjugating oligonucleotides to nanoparticles, for example thiol-modified and disulfide-modified oligonucleotides spontaneously bind to gold nanoparticles surfaces, di- and tri-sulphide modified conjugates, oligothiol-nanoparticle conjugates and oligonucleotide conjugates from Nanoprobes' phosphine-modified nanoparticles (see FIG. 2 of Fritzsche W, Taton T A, Nanotechnology 14 (2003) R63-R73 “Metal nanoparticles as labels for heterogeneous, chip-based DNA detection”).

FIG. 1 shows biotin integrated into a DNA or RNA molecule. When binding with a complementary probe occurs the duplex is labelled with an anti-biotin antibody which is tagged with a nanoparticle suitable for optical and electrical detection.

In one embodiment, both DNA or RNA strands may be biotinylated. The biotinylated target strand may be hybridized to oligonucleotide probe-coated magnetic beads. Streptavidin-coated gold nanoparticles may then bind to the captured target strand (Wang J, Xu D, Kawde A, Poslky R, Analytical Chemistry (2001), 73, 5576-5581 “Metal Nanoparticle-Based Electrochemical Stripping Potentiometric Detection of DNA hybridization”). The magnetic beads allow magnetic removal of non-hybridized DNA.

In the embodiment of the present invention wherein the one or more labels used in step (a) are different from the one or more labels used in step (b), the analyte(s) may be labelled, for example with fluorophore label(s) for step (a) and nanoparticle label(s) for step (b). The fluorophore is suitable for optical detection in step (a) and the nanoparticle is suitable for electrical detection in step (b). The analyte may be either labelled with the two different labels simultaneously or split into two aliquots and labelled separately. The optical and electrical data measurements are obtained either on one chip or on separate chips. The step of labelling the analyte(s) with different labels is represented in the flow diagram of FIG. 3 wherein the analyte is nucleic acid, the fluorophore is for optical detection in step (a) and the gold/silver nanoparticle is for detection in step (b).

A particularly preferred method for labelling the nucleic acid analyte(s) with different labels in this embodiment is represented in FIG. 3. This method employs a primer labelled with the label suitable for electrical detection. The primer binds to the target nucleic acid sequence and is extended using a suitable enzyme (reverse transcriptase for RNA and DNA polymerase for DNA). One or more of the nucleosides used for the primer extension are labelled with one or more labels for optical detection, for example fluorophores. Therefore, the extension step introduces one or more optical labels into the oligonucleotide. The final product of the extension step contains the two different labels.

### Optical Detection Method

The optical detection method is preferably selected from optical emission detection, optical absorbance detection, optical scattering detection, spectral shift detection, surface plasmon resonance imaging, and surface-enhanced Raman scattering from adsorbed dyes.

In a preferred embodiment, the optical detection method is optical emission detection and comprises the steps of irradiating the labelled analytes with light capable of exciting the labels and detecting the frequency and intensity of light emissions from the labels. The optical data of frequency and/or intensity can be used in step (c) of the method of the present invention to provide information on the identity and/or quantity of analytes present.

In this preferred embodiment, if a plurality of different labels is used to label different analytes, each label preferably has different frequency of emission. The type, composition, size, shape and roughness of the labels will determine the resonant frequency of the emission from the labels. Thus all of these properties of the labels can be changed to “tune” the frequency of emission to that desired. In this way, labels of the same material type, but differing dimensions (or the same dimensions, but differing material) can be employed in multiplexing methods.

In the present invention, the light employed in the optical detection method is not especially limited, provided that it is able to sufficiently excite the one or more labels. Typically the light to which the embedded labelled analyte is exposed is a laser light. The frequency of the light is also not especially limited, and UV, visible or infrared light may be employed.

In a preferred embodiment, when the labels are metal nanoparticles or metal nanoshells the light employed is white light.

In another preferred embodiment, when the labels are single molecules or quantum dots, the light employed is laser light.

Methods for carrying our other optical detection methods including optical absorbance detection, optical scattering detection, spectral shift detection, surface plasmon resonance imaging, and surface-enhanced Raman scattering from adsorbed dyes are well known in the art (Fritzsche W, Taton T A, Nanotechnology 14 (2003) R63-R73 “Metal nanoparticles as labels for heterogeneous, chip-based DNA detection”).

In a preferred embodiment the optical detection method is carried out on a chip.

### Electrical Detection Method

The electrical detection method is preferably selected from electrical resistive detection and electrochemical detection.

Electrical resistive detection methods are well known in the art (Fritzsche W, Taton T A, Nanotechnology 14 (2003) R63-R73 “Metal nanoparticles as labels for heterogeneous, chip-based DNA detection”)

Preferably, the electrical detection method is electrochemical detection. In one embodiment, the electrochemical detection comprises the steps of
(i) placing the labelled analytes into a solution comprising two electrodes whereby the one or more labels dissolve in the solution;
(ii) applying a deposition potential to the electrodes whereby the one or more labels deposit onto one of the electrodes; and
(iii) detecting the electrochemical signals from the electrode.

In step (i) the solution is not particularly limited provided that it is suitable for dissolving the one or more labels. Preferably the solution comprises an acid to cause dissolution of the one or more labels. This step usually destroys the analyte and the labels. Therefore, the optical detection method must be carried out before the electrochemical detection method.

In step (ii) a potential is applied in order to plate the labels on the electrode. The deposition time is not particularly limited but is preferably greater than 1 second, more preferably greater than 30 seconds, still more preferably more than 1 minute and most preferably 2 minutes.

The deposition step is typically a slow step, controlled by the relatively long time that it takes for the dissolved labels to diffuse through the solution and contact the electrode surface where the redox plating reaction occurs. Because the step is slow, the signal obtained may be relatively weak, and may not be suitable for measurement purposes. Therefore, in a preferred embodiment, step (ii) further comprises a step of applying a second potential to the electrodes to generate a second redox reaction of the labels deposited on the electrode. This generates a signal. The second redox reaction may be oxidation of the deposited labels. This second redox reaction is quicker since it is no longer diffusion controlled. This leads to a much stronger signal, i.e. greater sensitivity.

In one embodiment, wherein the labels are metal nanoparticles, for example gold, the second redox reaction is oxidation of the plated metal.

If a plurality of different labels is used to label different analytes, preferably each label has a different oxidation potential for the electrochemical detection method and, therefore, produces different signal peaks in the data obtained. For example, when metal nanoparticles are used as labels for different analytes, different metals with different oxidation potentials may be used for each analyte.

In step (ii) of the electrochemical detection method the deposition potential is preferably −0.1 V to −1.0 V, and more preferably −0.5 V to −0.8 V.

In the preferred embodiment of step (ii) wherein step (ii) further comprises a step of applying a second potential to the electrode to generate a redox reaction of the deposited labels, the second potential is +1.0 to +2.0 V, and preferably +1.2 V to +1.8 V.

In the preferred electrochemical detection method of the present invention the labels are preferably nanoparticles of a collection of species. This ensures that the signal produced in the electrochemical detection is large enough to be accurately and sensitively detected. When single molecule nanoparticles are used, this provides a very low current and therefore low sensitivity for detection.

In a preferred embodiment, the electrical detection method is carried out on a chip. This may be the same or a different chip used for the optical detection method. In the embodiment of the present invention where different label(s) are used in step (a) for optical detection and in step (b) for electrical detection, the optical and electrical detection may be carried on one chip when the analyte(s) have been labelled with the different labels simultaneously (see FIG. 2). Alternatively, where the analyte(s) have been separated into two aliquots and labelled separately they may then be combined after labelling for optical and electrical detection on one chip or optical and electrical detection may be carried out separately on two separate chips (see FIG. 2).

In the embodiment of the present invention where the analyte(s) is nucleic acid and the labelling step is performed using labelled primers and primer extension using labelled nucleosides (see FIG. 3), the labelled extended primer may be hybridised to a probe for optical and electrical detection (see FIG. 3). This is particularly advantageous because it allows the label(s) for electrical detection to be positioned in close proximity to the electrode for detection, as shown in FIG. 3.

Determining the Identity and/or Quantity of the Analyte from Both the Optical and Electrical Data

In step (c) of the method of the present invention, the identity and/or quantity of the analyte or plurality of analytes is determined from both the optical and electrical data obtained in step (b).

For example, when optical emission detection is used as the optical detection method the intensity of light emissions from the labels can be used to provide information on the identity and/or quantity of analytes present.

For example, when electrochemical detection is used as the electrical detection method the amount of label present can be quantified by voltammetry. Quantitative data can be obtained from the signal peaks by integration, i.e., determining the area under the graph for each signal peak produced.

Labelling DNA Analyte with Nanoparticle

RNA is reverse-transcribed, incorporating a nucleotide labelled with a nanoparticle, according to conventional techniques.

### Optical and Electrochemical Detection

Labels are excited with light of a given wavelength, and their emission is detected at a predetermined wavelength, according to conventional methods.

Electrochemical detection is then carried out on the labelled analyte from the optical detection method. The labelled analyte is dissolved in an acidic solution. Electrodes are inserted into the solution and a deposition potential of −0.8 V. After a deposition time of two minutes a second potential of +1.2 V is applied to oxidise the deposited nanoparticles. Electrochemical signals are detected.

The present invention will be described further by way of example only.

### EXAMPLES

### Example 1

### Protocols

### Cleaning of Gold Electrodes

Gold electrodes were cleaned using an electrochemical pulse method at 1.4 V (vs Ag/AgCl reference electrode) in phosphate buffer saline (PBS) for 30 s. Electrodes were then washed for 5 min with ultra-pure water at room temperature. Electrodes were dried under a stream of nitrogen for 1 min at room temperature.

Immobilization of 75-mer Thiol-Modified ssDNA (HCV Probe)

Prior to immobilisation, the disulfide protecting group was removed from the thioated oligonucleotide using 5 mM TCEP (Tris(2-carboxyethyl)phosphine hydrochloride) in PBS for 30 min, followed by purification using a MicroSpin™ G-25 column. Oligonucleotides (HCV probes=5′-GGC AAT TCC GGT GTA CTC ACC GGT TCC GCA GAC CAC TAT GGC TCT CCC GGG AGG GGG GG-3′[′]=SH) (10 μM 75-mer thiol-modified ssDNA) were incubated on cleaned gold electrode in PBS (10 mM, 137 mM NaCl, 2.7 mM KCl at pH 7.4) for 16 h at 30° C. Electrodes were washed three times with PBS, NaCl (1 M) followed by PBS for 10 min each. Electrodes were dried under a stream of nitrogen for 1 min at room temperature. Prior to use, electrodes were stored in 2×SSC buffer at 4° C.

### Electrochemical and Optical Characterization

Electrochemical Impedance Spectroscopy was performed in 2×SSC containing 10 mM [Fe(CN)6]^{3-/4-} (electrochemical buffer (EB)) using an ac voltage 10 mV superimposed on a DC voltage 0.24 V vs Ag/AgCl reference in the frequency window 100 KHz-100 mHz. Electrodes were then washed with water (1 min) at room temperature and dried under a stream of nitrogen (1 min). For hybridization with HCV target, electrodes were incubated for 2 hours in 2×SSC buffer at 55° C. with 100 nM DNA target (A or B):

A: complementary HCV target =
5′-CCC CCC CTC CCG GGA GAG CCA TAG TGG TCT GCG GAA
CCG G-3′ [5′] = Cy3)
B: non-complementary HCV target =
5′-AGT GTT GAG GGC CGT AAG CGT GTT GTG TCC GAC GCT
GCC TGC GCA CTG CCG GTG CGT GTC GTC CCA CGG TAT
TTG-3′, [5′] = Cy3

After hybridisation, electrodes were washed with 2×SSC followed by 0.2×SSC for 10 min at room temperature. Fluorescence was measured in a microarray scanner (Tecan LS Reloaded) using excitation at 534 nm and emission 570 nm (see below). Stripping of target from electrode was achieved by washing for 3 mins at 90° C. in water.

### Results

To assess the outcome of electrochemical and optical detection of a single hybridisation experiment, the detection of the electron transfer resistance (Ret) was performed by electrochemical impedance spectroscopy (see FIG. 1) and fluorescence intensity measurements (see FIG. 2) using a Tecan LS reloaded microarray scanner. A clear increase of Ret by 10 kΩ and fluorescence intensity could be seen after hybridisation of a complementary target DNA (see Electrode 1). The fluorescence intensity values of electrode only with probes are in the range of 27±57 a.u. The incubation with a non-complementary target led to clearly diminished signal changes (see Electrode 2) and can be clearly discriminated from the complementary target event. Note, the assessment of binding effects of different targets were done with different electrodes to exclude cross contamination.

| | Electrode + | | Non- |
|---|---|---|---|
| | probes | Complementary | complementary |
| Electrode 1 | (no target) | target | target |
| Fluorescence [a.u.] | 27 ± 57* | 2743 ± 891 | nd |
| Ret [kΩ] | 25.29 | 36.3 | nd |

*fluorescence intensity values of probe covered electrodes where done with separate electrodes to exclude any interferences of the detection process on the actual experiment

| | Electrode + | | |
|---|---|---|---|
| | probes | Complementary | Non-complementary |
| Electrode 2 | (no target) | target | target |
| Fluorescence | 27 ± 57* | nd | 78 ± 46 |
| Ret [kΩ] | 7.97 | nd | 6.2 |

*fluorescence intensity values of probe covered electrodes where done with separate electrodes to exclude any interferences of the detection process on the actual experiment

1. A method for detecting one or more analytes, wherein the analyte is labelled with one or more labels relatable to the analyte, which method comprises:
a) performing an optical detection method on the labelled analyte to obtain optical data from the one or more labels;
b) performing an electrical detection method on the labelled analyte to obtain electrical data from the one or more labels; and
c) determining the identity and/or quantity of the analyte from both the optical and electrical data.

2. A method according to claim 1, wherein the one or more labels are suitable for optical and electrical detection, and the one or more labels in step (a) are the same as the one or more labels in step (b).

3. A method according to claim 2, wherein before step (a), the method further comprises the step of labelling the analyte with the one or more labels to form the labelled analyte.

4. A method according to claim 1, wherein the one or more labels in step (a) are suitable for optical detection and the one or more labels in step (b) are suitable for electrical detection and the one or more labels in step (a) are different from the one or more labels in step (b).

5. A method according to claim 4, wherein before step (a), the method further comprises the step of labelling the analyte with the one or more labels in step (a) and the one or more labels in step (b) either simultaneously or separately to form the labelled analyte.

6. The method of claim 1, wherein the one or more analytes comprises a plurality of analytes, wherein each different analyte is labelled with one or more different labels relatable to the analyte, which method comprises:
a) performing an optical detection method on a plurality of labelled analytes to obtain optical data from the labels;
b) performing an electrochemical detection method on the plurality of labelled analytes to obtain electrical data from the labels; and
c) determining the identity and/or quantity of the plurality of analytes from both the optical and electrical data.

7. A method according to claim 6, wherein the one or more labels are suitable for optical and electrical detection and the one or more labels for each analyte in step (a) are the same as the one or more labels for each analyte in step (b).

8. A method according to claim 7, wherein before step (a), the method further comprises the step of labelling the plurality of analytes with the one or more labels to form the plurality of labelled analytes.

9. A method according to claim 6, wherein the one or more labels in step (a) are suitable for optical detection and the one or more labels in step (b) are suitable for electrical detection and the one or more labels for each analyte in step (a) are different from the one or more labels for each analyte in step (b).

10. A method according to claim 9, wherein before step (a), the method further comprises the step of labelling the plurality of analytes with the one or more labels in step (a) and the one or more labels in step (b) either simultaneously or separately to form the plurality of labelled analytes.

11. A method according to claim 1, wherein the labels are selected from nanoparticles, single molecules, chemiluminescent enzymes and fluorophores.

12. A method according to claim 11, wherein the labels are nanoparticles comprising a collection of molecules and/or atoms.

13. A method according to claim 12, wherein the nanoparticles are selected from metals, metal nanoshells, metal binary compounds and quantum dots.

14. A method according to claim 13, wherein the nanoparticles are metal compounds selected from CdSe, ZnS, CdTe, CdS, PbS, PbSe, Hgl, ZnTe, GaAs, HgS, CdAs, CdP, ZnP, AgS, InP, GaP, GaInP, and InGaN.

15. A method according to claim 13, wherein the nanoparticles are selected from gold, silver, copper, cadmium, selenium, palladium and platinum.

16-18. (canceled)

19. A method according to claim 6, wherein the one or more labels for each different analyte have different physical properties.

20. A method according to claim 19, wherein the physical properties are selected from one or more of size, shape and surface roughness.

21. A method according to claim 6, wherein the labels for each different analyte have different compositions.

22. A method according to claim 6, wherein the labels for each different analyte are of different types.

23. A method according to claim 1, wherein the optical detection method is selected from optical emission detection, optical absorbance detection, optical scattering detection, spectral shift detection, surface plasmon resonance imaging, and surface-enhanced Raman scattering from adsorbed dyes.

24. A method according to claim 23, wherein the optical detection method is optical emission detection and comprises the steps of irradiating the labelled analytes with light capable of exciting the labels and detecting the frequency and intensity of light emissions from the labels.

25. A method according to claim 24, wherein the light is laser light.

26. A method according to claim 24, wherein the light is selected from infra-red light, visible light and UV light.

27. A method according to claim 26, wherein the light is white light.

28. A method according to claim 1, wherein the electrical detection method is selected from electrical resistive detection and electrochemical detection.

29. A method according to claim 28, wherein the electrical detection method is electrochemical detection and comprises the steps of
(i) placing the labelled analytes into a solution comprising two electrodes whereby the one or more labels dissolve in the solution;
(ii) applying a deposition potential to the electrodes whereby the one or more labels deposit onto one of the electrodes; and
(iii) detecting the electrochemical signals from the electrode.

30. A method according to claim 29, wherein the deposition potential is −0.1 V to −1.0 V.

31. A method according to claim 30, wherein the potential is an AC voltage superimposed on a DC voltage.

32. A method according to claim 31, wherein the AC voltage is about 10 mV superimposed on a DC voltage of about 0.24 V.

33. A method according to claim 29, wherein step (ii) further comprises a step of applying a second potential to the electrodes to generate a redox reaction of the deposited labels.

34. A method according to claim 33, wherein the second potential is from +1.0 to +2.0 V.

35. A method according to claim 33, wherein the redox reaction is oxidation of the deposited labels.

36. A method according to claim 1, wherein the analyte comprises one or more compounds selected from a cell, a protein, a polypeptide, a peptide, a peptide fragment, an amino acid, DNA and RNA.

37. A method according to claim 36, wherein the analyte is DNA or RNA and the step of labelling the analyte or plurality of analytes with the one or more labels in step (a) and the one or more labels in step (b) comprises the following steps:
i. binding a primer to the DNA or RNA, wherein the primer is labelled with one or more labels suitable for electrical detection in step (b); and
ii. extending the primer enzymatically with nucleosides, wherein one or more the nucleosides is labelled with one or more labels suitable for optical detection in step (a).
